(19) United States
(12) Patent Application Publication
(10) Pub. No.: US 2010/0094427 A1
(43) Pub. Date: Apr. 15, 2010

(54) Movable Intervertebral Disc Prosthesis

(76) Inventor: Rudolf Bertagnoli, Straubing (DE)

Correspondence Address: ROBERT W. BECKER & ASSOCIATES, 707 HIGHWAY 333, SUITE B, TIJERAS, NM, 87059-7507, US

(21) Appl. No.: 12/578,996

(22) Filed: Oct. 14, 2009

(30) Foreign Application Priority Data
Oct. 14, 2008 (DE) 10 2008 051 588.4

(51) Int. Cl. A61F 2/44

(52) U.S. Cl. 623/17.16

(57) ABSTRACT

A movable intervertebral disc prosthesis having a pivot point and adapted to be implanted between vertebra bodies to form a vertebrae segment. In an implanted state three-dimensional movement axes of the vertebrae segment intersect in the pivot point. The configuration of the prosthesis is such that a deliberate displacement or shifting of the pivot point can be carried out prior to, during and/or after the implantation.

The instant application should be granted the priority date of Oct. 14, 2008 the filing date of the corresponding German patent application DE 10 2008 051 588.4

### BACKGROUND OF THE INVENTION

The present invention relates to a movable intervertebral disc prosthesis or artificial disc having a pivot point, whereby in the implanted state three-dimensional movement axes of a vertebrae segment intersect.

The implantation of movable intervertebral disc prostheses requires great care, which relates to the pivot point. The natural pivot point, via which an entire vertebrae segment is moved, normally lies in the rear third of the intervertebral disc. Furthermore, the natural pivot point moves on a plane, essentially following a circle, depending upon the positions or orientations of the vertebra bodies of the patient.

Conventional movable intervertebral disc prostheses define a pivot point that in the implanted state assumes a fixed position. If this pivot point does not lie in the equilibrium plane of the natural movement sequence, premature wear of the prosthesis, and possibly also further wear of the movement segment, can occur.

DE 103 61 772 A1 is intended to take this drawback into account with an intervertebral disc prosthesis by providing a floatingly mounted, hemispherical intervertebral disc between two artificial end plates that are connected to the adjoining vertebra bodies; the disc can carry out translational movements between the artificial end plates. Although the rotational center point of the rotational and bending movements is shifted with the movement of the spine, this is, however, effected in an uncontrolled manner that twists the prosthesis unnaturally and in so doing damages the remaining joints.

It is an object of the present invention to develop a prosthesis of the aforementioned general type via which the implantation can be simplified with regard to an as precise a placement of the pivot point as possible.

### BRIEF DESCRIPTION OF THE DRAWINGS

This object, and other objects and advantages of the present application, will appear more clearly from the following specification in conjunction with the accompanying schematic drawings, in which:

FIG. 1 shows a vertebrae segment,

FIGS. 2a and 2b are cross-sectional views through one exemplary embodiment of the present invention, and

FIG. 3 shows a shifting or adjusting mechanism.

### SUMMARY OF THE INVENTION

The object of the present application is inventively realized by a movable intervertebral disc prosthesis or artificial disc having a pivot point and adapted to be implanted between vertebra bodies to form a vertebrae segment, wherein in an implanted state, three-dimensional movement axes of the vertebrae segment intersect in the pivot point, and wherein the intervertebral disc prosthesis is configured such that a deliberate displacement or shifting of the pivot point can be carried out prior, during and/or after implantation.

By means of this possibility for adjusting or post adjusting the prosthesis, the position of the pivot point can be deliberately placed or corrected, and hence extensively optimally disposed, without altering the position of the prosthesis and the positive anchoring condition. Application occurs on conventional and tested prosthesis systems.

The present invention has the advantage that the prosthesis is adjusted for optimizing the position of the pivot point, whereby the prosthesis components or modules that define the pivot point do not twist in an uncontrolled manner, as is the case with the aforementioned state of the art.

Modular prostheses are generally used, with which the prosthesis modules that define the pivot point are configured so as to be shiftable or displaceable in a translatory manner and in one or two dimensions relative to the natural end plates of the adjoining vertebra bodies. In this way, the pivot point can to a large extent be calibrated in such way that the entire movement segment of the spine can be held in a stable manner, i.e. in equilibrium. With knowledge of the anatomy of the patient, the positioning of the pivot point can be effected prior to the implantation. It is expedient to select a configuration of the prosthesis that also permits an adjustment or setting of the pivot point position during the operation, which also provides the possibility of undertaking correction after an operation has taken place or in a repair operation.

The components of a modular prosthesis are configured to be movable or displaceable relative to one another. By standardizing the components, the replacement or the combination adapted for the respective patient can be put together easily and rapidly. In this connection, basic components, such as artificial end plates, can have a uniform configuration.

One advantageous prosthesis is comprised of two artificial end plates that between them accommodate an intervertebral module that is movable about the pivot point, wherein the intervertebral module is configured to be displaceable parallel to the planes of the end plates.

Pursuant to a straightforward solution from a manufacturing standpoint, the movable intervertebral module is comprised of two components, for example a hemisphere/socket pair. Both components are either configured to be displaceable as a unit, or pursuant to a straightforward solution only one of the two components is displaceable relative to the pertaining end plate, while the other component is mounted in a floating manner with the second end plate in such a way that it is carried along or displaced with translatory displacement or shifting of the first component.

An adjustment mechanism that is straightforward from a manufacturing standpoint is a screw adjustment, according to which the intervertebral module or only one of its components can be shifted or displaced in a one-dimensional or lineal manner relative to reference modules, for example artificial end plates. This adjustability is, for example, selected in the direction ventral-dorsal of the patient if the optimum lateral position of the pivot point can be achieved by symmetrical configuration of the prosthesis. This solution, in the event of an incorrect setting, which appears after the operation, permits a post adjustment by actuating the adjusting screw, which can be carried out with little post operative effort.

A two-dimensional translation is preferably provided that enables the pivot point of the prosthesis to be adjusted not only in the direction ventral/dorsal but also laterally in an optimum manner with respect to the pivot point position of the natural movement segment.

Translatory adjustment possibilities by means of adjustment screws can be actuated manually or electrically via induction coils. In the latter case, corrections can also be effected externally, i.e. without operative intervention or invasion.

All technically possible solutions for the displacement or shifting of translatorially movable prosthesis modules can be utilized that can be realized from a size standpoint for intervertebral disc implants.

In addition to the rotational movements, a natural vertebrae segment also carries out translatory movements perpendicular to the vertical axis. The pivot point of the natural movement apparatus, at various vertebra body positions, moves along a plane, approximately following a circle. To reproduce this natural movement of the pivot point, pursuant to a further embodiment of the invention a servomotor is proposed via which the translatory movements of the pivot point can be imitated in conformity with the natural movement. Movement sensors on vertebra bodies and/or muscles can deliver the necessary signals to the appropriately programmed servomotor.

The technical realization of solutions for the translatory shifting or displacement of module components of an intervertebral disc prosthesis is to be sought in the pertaining technology or specialty. The important thing here is the concept of an adjustment of the artificial pivot point as precisely as possible by means of a one time, or repeated or continuous, translatory shifting or displacement of prosthesis modules.

Further specific features of the present invention will be described in detail subsequently.

### DESCRIPTION OF SPECIFIC EMBODIMENTS

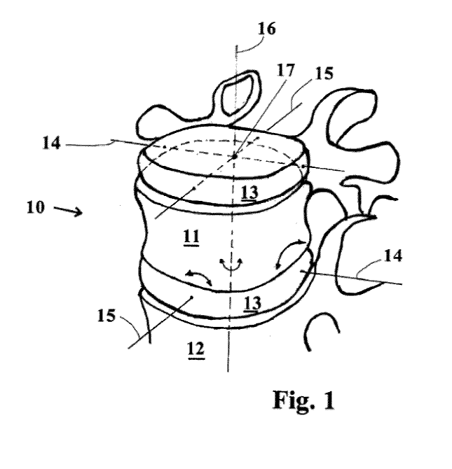

Referring now to the drawings in detail, FIG. 1 illustrates a vertebrae segment 10 composed of two vertebra bodies 11, 12 and an intervertebral disc 13. When the patient moves, the vertebra bodies 11, 12 of the vertebrae segment 10 carry out or execute relative movements about an extension/bending axis 14 and a lateral tilting axis 15 as well as pivoting or rotating about a vertical axis 16. These axes 14-16 intersect in a pivot point 17. This natural pivot point 17 ends up approximately in the rear third of the intervertebral disc.

If the natural intervertebral disc 13 is replaced by a movable implant, an artificial pivot point is reproduced to obtain the moveability of appropriately moveably configured implant components or modules.

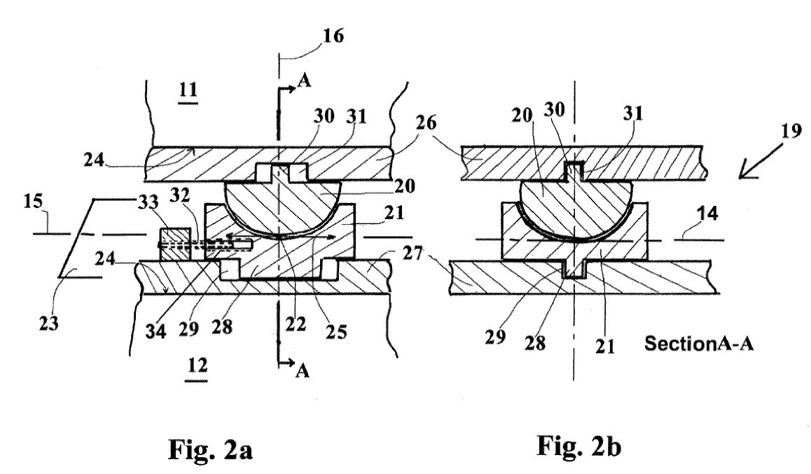

A straightforward example is a hemisphere/socket prosthesis or artificial disc, as illustrated in longitudinal section in the direction ventral-dorsal in FIG. 2a. FIG. 2b shows a longitudinal section A-A taken transverse to FIG. 2a.

Within the socket 21, the hemisphere 20 has a three dimensional moveability, with an artificial pivot point 22.

The correct placement of the artificial pivot point 22 on a plane 23 parallel to the natural end plates 24 of the vertebra bodies 11, 12 plays an important role for the stability of the vertebral or spinal column.

Pursuant to the exemplary embodiment of FIGS. 2a and 2b, an adjustment possibility is provided via which the position of the artificial pivot point 22 can be shifted or displaced translatorially in the ventral-dorsal direction 25. The modularly configured prosthesis or artificial disc 19 is composed of two artificial end plates 26, 27 that are fixedly connected with a respective one of the vertebra bodies 11 or 12, and which accommodate the hemisphere 20 or the socket 21 respectively with play.

The socket 21 has an elongated, ventrally-dorsally directed raised portion or rib 28, which extends into a slotted recess 29 of the lower artificial end plate 27 in such a way that the rib 28 is longitudinally displaceable in the recess 29. In a similar manner, a raised portion or rib 30 of the hemisphere 20 extends into a corresponding recess 31 in the upper artificial end plate 26 in such a way that hemisphere 20 and socket 21 can be moved in common, and hence the pivot point 22 can be moved in the ventral-dorsal direction.

In order to adjust the position of the pivot point 22, a screw 32 is rotatably mounted in a stub 33 that is fixed on the lower artificial end plate 27. The screw 32 extends into a threaded bore 34 of the socket 21 in such a way that when the screw 32 is rotated, the socket 21, and hence the rib 28, can moved back and forth within the recess 29. The floatingly held hemisphere 20 is pulled along with the socket 21 so that the pivot point 22 can be brought into the desired ventral-dorsal position by means of adjustment at the screw 32.

With knowledge of the precise anatomy of the point of operation of the patient, the adjustment can be effected prior to implantation of the intervertebral disc prosthesis 19. Generally, the exact adjustment of the position of the pivot point 22 is undertaken during the implantation. With the embodiment described, an adjustment is also possible at a later point in time, for example if an imbalance in the vertebra segment is discovered. For this purpose, merely a minimally invasive operation is required for the rotation of the screw 32.

The intervertebral disc prosthesis 19 can be produced in a modular, preferably a standardized modular, construction, whereby the artificial end plates 26 and 27 that are provided with the recesses 29, 31 are permanently fixed on the adjoining vertebra bodies 11, 12. The movable intervertebral module can be exchanged or replaced.

To facilitate illustration, an intervertebral disc prosthesis having translatory displaceability of the pivot point 22 in only one direction 25 is described.

However, the prosthesis of the present application covers all possible constructions and mechanisms for the shifting or displacement of the pivot point in one or two directions or on a plane.

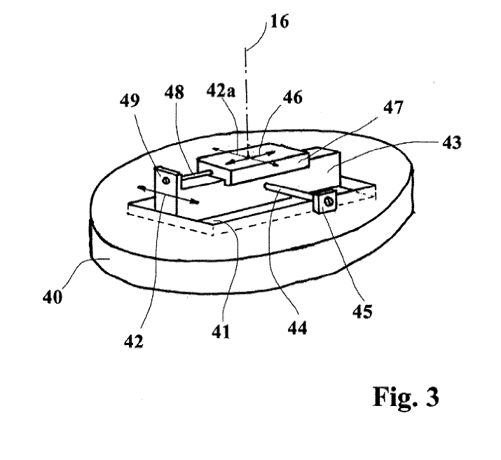

Also to facilitate illustration, a mechanism that is displaceable in two directions is illustrated diagramatically in FIG. 3 to show the principle thereof.

A plate 40 having a rectangular recess 41 serves as a reference plane, with a movable raised portion or rib 43 that is movable in a first direction 42 being placed in the recess 41. The translatory movement in this first direction 42 is effected by means of a screw 44, which is supported on a stub 45 that forms a unit with the reference plate or plane 40.

The rib 43 that is movable in the first direction 42 forms the reference support for the second direction of movement 46 and supports a carriage 47, which is seated on the rib 43 in such a way as to be displaceable along the rib. The carriage 47 is moved by means of a second screw 48, which in turn is supported on a stub 49 that is unitarily connected with the rib 43.

The carriage 47 can thus be moved back and forth in the first direction 42a by means of the rib 43, while it can be moved back forth on the rib 43 in the second, transverse direction 46.

Applied to an intervertebral disc prosthesis, the reference plate 40 represents an artificial end plate and the carriage 47 could be designed as a socket. The pivot point 22 of the embodiment of FIG. 2a is therewith translatorially displaceable within a rectangular plane, so that by means of the two adjustment screws 44 and 48, the pivot point 22 can be precisely placed in the implanted state of the intervertebral disc prosthesis.

Techniques are conceivable that enable an electronic or electro inductive control of, for example, the screws, as a result of which a post-implantation adjustment of the pivot point can be carried out without direct operative intrusion. Modern electronics also make possible control devices that undertake a shifting or displacement of the pivot point during the movement of the patient. The artificial pivot point 22, which according to the example of FIG. 3 can move on a rectangular plane, will be able to cover the natural circular surface by design of the movement technology. This has the advantage that with appropriate electronics, an automatic continuous displacement of the pivot point location can be carried out as a function of the position of the spine and the movement of the patient.

The specification incorporates by reference the disclosure of German priority document DE 10 2008 051 588.4 filed Oct. 14, 2008.

The present invention is, of course, in no way restricted to the specific disclosure of the specification and drawings, but also encompasses any modifications within the scope of the appended claims.

What I claim is:

1. A movable intervertebral disc prosthesis (19), having a pivot point (22) and adapted to be implanted between vertebra bodies (11, 12) to form a vertebrae segment (10), wherein in an implanted state three-dimensional movement axes of the vertebra segment (10) intersect in the pivot point (22), and wherein said prosthesis (19) has a configuration such that a deliberate displacement or shifting (25, 42a, 46) of the pivot point (22) can be carried out in at least one of the states consisting of prior to, during and after implantation.

2. Intervertebral disc prosthesis according to claim 1, which includes means (32; 44, 48) for carrying out the displacement or shifting (25; 42a, 46) of the pivot point (22) relative to fixed components (26, 27; 40).

3. Intervertebral disc prosthesis according to claim 2 wherein said means for carrying out the displacement or shifting (25 42a, 46) of the pivot point (22) are adjustment screws (32, 44, 48).

4. Intervertebral disc prosthesis according to claim 1, which includes induction coils configured to externally control the position of the pivot point (22).

5. Intervertebral disc prosthesis according to claim 1, wherein a servomotor is associated with said prosthesis (19) for continuously adapting the position of the pivot point (22) to conditions in a human body in which said prosthesis (19) is implanted.

6. Intervertebral disc prosthesis according to claim 1, wherein said prosthesis (19) is a modular prosthesis comprised of module components (26, 27), which are adapted to be fixedly anchored with said vertebra bodies (11, 12), as well as movable module components (20, 21; 47) that are adapted to be translatorially displaceable relative to said first mentioned module components (26, 27).

7. Intervertebral disc prosthesis according to claim 6, wherein said movable module components (20, 21, 47), are replaceable.

8. Intervertebral disc prosthesis according to claim 6, wherein said fixedly anchorable module components are two artificial end plates (26, 27), and wherein said movable module components are an intervertebral module (20, 21) that is accommodated between said end plates (26, 27) so as to be displaceable approximately parallel to said end plates.

9. Intervertebral disc prosthesis according to claim 8, wherein said movable intervertebral module (20, 21) is comprised of at least two components, further wherein a first one of said components (21) is displaceable relative to an associated one of said artificial end plates (27) and wherein a second one of said components (20) is mounted at least partially in a floating manner between the other one of said end plates (26) and said first component (21) of said intervertebral module.

10. Intervertebral disc prosthesis according to claim 9, wherein said movable intervertebral module (20, 21) is replaceably configured.
